(19) United States
(12) Patent Application Publication
(10) Pub. No.: US 2004/0030247 A1
(43) Pub. Date: Feb. 12, 2004

(54) Apparatus, systems and methods for facilitating multiple imaging procedures for a patient

(76) Inventors: Mark Trocki, Cheswick, PA (US); George J. Misic, Allison Park, PA (US)

Correspondence Address: GREGORY L BRADLEY, MEDRAD INC, ONE MEDRAD DRIVE, INDIANOLA, PA, 15051

(21) Appl. No.: 10/384,073

(22) Filed: Mar. 7, 2003

Related U.S. Application Data
(60) Provisional application No. 60/363,918, filed on Mar. 13, 2002.

(51) Int. Cl. A61B 6/00

(52) U.S. Cl. 600/431

(57) ABSTRACT

A method of performing multiple imaging procedures on a patient includes injecting contrast fluid from a removable contrast fluid container into the patient, generating at least a first contrast-enhanced image of the patient using a first imaging system, disconnecting the removable contrast fluid container from an injector system, moving the patient and the removable contrast fluid container from the first imaging system to a second imaging system, generating an image of the patient using the second imaging system, and moving the patient and the removable contrast fluid container from the second imaging system to the first imaging system.

### CROSS REFERENCE TO RELATED APPLICATION

This application claims the benefit of U.S. Provisional Application Serial No. 60/363,918, filed on Mar. 13, 2002, the contents of which are incorporated herein by reference.

### BACKGROUND OF THE INVENTION

The present invention relates generally to an apparatus, systems and methods for facilitating or performing multiple imaging procedures for or on a patient and, more particularly, to an apparatus, systems and methods for facilitating multiple angiographic/fluoroscopic and magnetic resonance imaging procedures, including contrast-enhanced versions of those procedures, for or on a patient.

A number of diagnostic imaging techniques are currently available for treating patients. These include angiographic, fluoroscopic, computed tomography (CT), magnetic resonance (MRI/NMR) and ultrasound imaging techniques. The images produced by these imaging techniques are often enhanced by injecting contrast fluid into the patient. The contrast fluid improves the images by increasing the contrast between the tissue or organ being imaged and the surrounding tissue. Typically, the contrast fluid is injected by means of a powered injector specifically designed for use with a particular diagnostic imaging systems.

Typically, the diagnostic imaging systems (including their attendant powered injector systems) are situated in dedicated suites in, for example, a hospital or specialized clinic. These imaging systems are typically located in different areas of the hospital and are therefore not readily accessible to a patient requiring more then one type of imaging procedures. Consequently, if a patient could benefit from more then one imaging technique, the patient will typically need to be moved to and among the requisite imaging suites. Especially during emergency situations, the time and effort required to shuttle a patient to and among multiple diagnostic imaging suites may have a significant affect on a health care provider's ability to quickly diagnose and treat an illness.

To alleviate the drawback of having scattered imaging suites in a medical facility, some leading medical research hospitals have recently placed two diagnostic imaging systems—an angiographic/fluoroscopic imaging system and a magnetic resonance imaging (MRI) system—in a single suite (sometimes referred to as an “XMR” suite). While these XMR suites have potentially reduced the time and effort required to move a patient between an “angio” suite and an MRI suite, they have not addressed the problem of shuttling a powered medical injector and/or contrast fluid source with the patient between the “angio” and MRI systems within the XMR suite. This problem is exacerbated for MRI systems because conventional injectors designed for use with angiographic, CT and ultrasound imaging systems, for example, are not compatible with MRI systems. See U.S. Pat. No. Re. 36,648, the contents of which are incorporated herein by reference.

There is thus a need for a medical injector and a contrast fluid container, such as a syringe or a pressure jacket assembly including a syringe, that can be disconnected from the injector and moved with a patient between multiple imaging systems for facilitating multiple imaging procedures.

### SUMMARY OF THE INVENTION

The present invention provides an apparatus, systems and methods for performing or facilitating multiple imaging procedures, including contrast-enhanced imaging procedures, on or for a patient. In a first embodiment, the present invention provides a method of performing multiple imaging procedures on or for a patient including injecting contrast fluid from a removable contrast fluid container into the patient, generating at least a first contrast-enhanced image of the patient using a first imaging system, disconnecting the removable contrast fluid container from an injector system, moving the patient and the removable contrast fluid container from the first imaging system to a second imaging system, generating an image of the patient using the second imaging system, and moving the patient and the removable contrast fluid container from the second imaging system to the first imaging system.

In a preferred embodiment, the method further includes connecting the removable contrast fluid container to the injector system, injecting contrast fluid from the removable contrast fluid container into the patient, and generating at least a second contrast-enhanced image of the patient using the first imaging system.

In a most preferred embodiment, the first imaging system is an angiographic (which includes, but is not limited to, fluoroscopic and radiographic techniques) imaging system, the second imaging system is a magnetic resonance imaging system and/or the injector system is an angiographic injector system. However, in alternate embodiments, one or more of the first and second imaging systems and the injector system is selected from, but not limited to, computed tomography and ultrasound imaging and injector systems.

Further, in a preferred embodiment, the removable contrast fluid container comprises a syringe that is removably connected to an injector. In a most preferred embodiment, the syringe is at least partially surrounded by a pressure jacket to prevent the syringe from bursting under angiographic pressures, which often exceed 1000 p.s.i. and may reach pressures of 1200 p.s.i. Preferably, the injector, syringe and/or pressure jacket are of the front-loading variety.

A fluid path, such as a connector tube, is connected at one end to the removable contrast fluid container, such as the fluid discharge end of the syringe, and at the other end to a catheter disposed in a patient. In a preferred embodiment, the fluid path remains connected to the removable contrast fluid container and the catheter after the removable contrast fluid container is disconnected from the injector.

In a preferred embodiment, the fluid path includes a stopcock that is activated to close and open the fluid path to the patient. Preferably, the fluid path to the patient is closed prior to disconnecting the removable contrast fluid container from the injector system. Further, the fluid path to the patient is preferably opened after the removable contrast fluid container is reconnected to the injector system.

The removable contrast fluid container is preferably formed of non-ferromagnetic material and is designed to not substantially interfere with an electromagnetic field of the magnetic resonance imaging system. These features are desired to substantially prevent the removable contrast fluid container from interfering with the operation of a magnetic resonance imaging system, which can be manifested by artifacts created in the images generated by the magnetic resonance imaging system.

In an alternate embodiment, and preferably when the contrast fluid is compatible with different imaging procedures, the removable contrast fluid container may be compatible with powered injectors specifically designed for the respective imaging systems. For example, the removable contrast fluid container may be connected to an angiographic injector for a first imaging procedure on a patient and then removed and connected to a CT injector for a second imaging procedure.

In a second embodiment, the present invention provides an injector system including an injector, a front-loading pressure jacket assembly and a syringe. Preferably, the injector includes a housing and a retaining mechanism associated with the housing. The retaining mechanism includes at least one retaining flange. The front-loading pressure jacket assembly includes a pressure jacket having a front end and a rear end, the rear end adapted to receive a syringe inserted therein, and a mounting member operably associated with the rear end of the pressure jacket. The mounting member includes at least one mounting flange adapted to releasably engage the at least one retaining flange of the retaining mechanism.

The mounting member is preferably formed of a non-ferromagnetic material, such as stainless steel, and the pressure jacket is preferably formed of a polymeric material, such as polycarbonate. The syringe includes a body defining a rear end and a fluid discharge end, and a plunger movably disposed within the body. The syringe is preferably formed of a polymeric material, such as polypropylene, polyethylene or TPX.

In a third embodiment, the present invention provides a system for performing multiple imaging procedures on or for a patient. The system includes a first imaging system, a second imaging system, and an injector system. In a preferred embodiment, the first imaging system is an angiographic imaging system, the second imaging system is a magnetic resonance imaging system and the injector system is an angiographic injector system. In alternate embodiments, more than two imaging systems, for example, CT, ultrasound and PET imaging systems, may be used.

The injector system preferably includes an injector, a front-loading pressure jacket, and a syringe. Preferably, the injector includes a housing and a retaining mechanism associated with the housing. The retaining mechanism includes at least one retaining flange. The front-loading pressure jacket assembly includes a pressure jacket having a front end and a rear end, the rear end adapted to receive a syringe inserted therein, and a mounting member operably associated with the rear end of the pressure jacket. The mounting member includes at least one mounting flange adapted to releasably engage the at least one retaining flange of the retaining mechanism. The mounting member is preferably formed of a non-ferromagnetic material, such as stainless steel, and the pressure jacket is preferably formed of a polymeric material, such as polycarbonate. The syringe includes a body defining a rear end and a fluid discharge end, and a plunger movably disposed within the body. The syringe is preferably formed of a polymeric material, such as polypropylene, polyethylene or TPX.

By providing an apparatus, systems and methods for moving or shuttling a medical injector and/or a contrast fluid container, such as a syringe or bulk contrast container, with patients between or among multiple (i.e., two or more) imaging systems, which may be included in a single suite, such as an XMR suite, the present invention provides or facilitates prompt and efficient diagnostic imaging, including contrast-enhanced imaging, of patients, thereby improving a health care provider's ability to more quickly diagnose and treat illness.

The present invention, along with the attributes and attendant advantages thereof, will best be appreciated and understood in view of the following detailed description taken in conjunction with the accompanying drawings.

### BRIEF DESCRIPTION OF THE DRAWINGS

FIG. 1 is a diagrammatic view of a suite including two different imaging systems.

FIG. 2 is a perspective view of a powered injector that may be used with or in the present invention.

FIG. 3 is an exploded perspective view of a preferred embodiment of the pressure jacket assembly of the present invention;

FIG. 4 is a perspective view of an alternate powered injector that may be used with or in the present invention.

FIG. 5 is an exploded perspective view of the pressure jacket assembly used with the powered injector shown in FIG. 4.

FIG. 6 is an exploded perspective view of an alternate powered injector and pressure jacket assembly that may be used with or in the present invention.

FIG. 7 is an exploded perspective view of an alternate powered injector and front-loading syringe that may be used with or in the present invention.

### DETAILED DESCRIPTION OF THE INVENTION

The present invention is discussed herein primarily with respect to an angiographic/fluoroscopic imaging system and a magnetic resonance imaging system placed in a single suite (sometimes referred to as an “XMR” suite). However, other and additional imaging systems, including CT, PET and ultrasound imaging systems, can be included in the suite depending on the application.

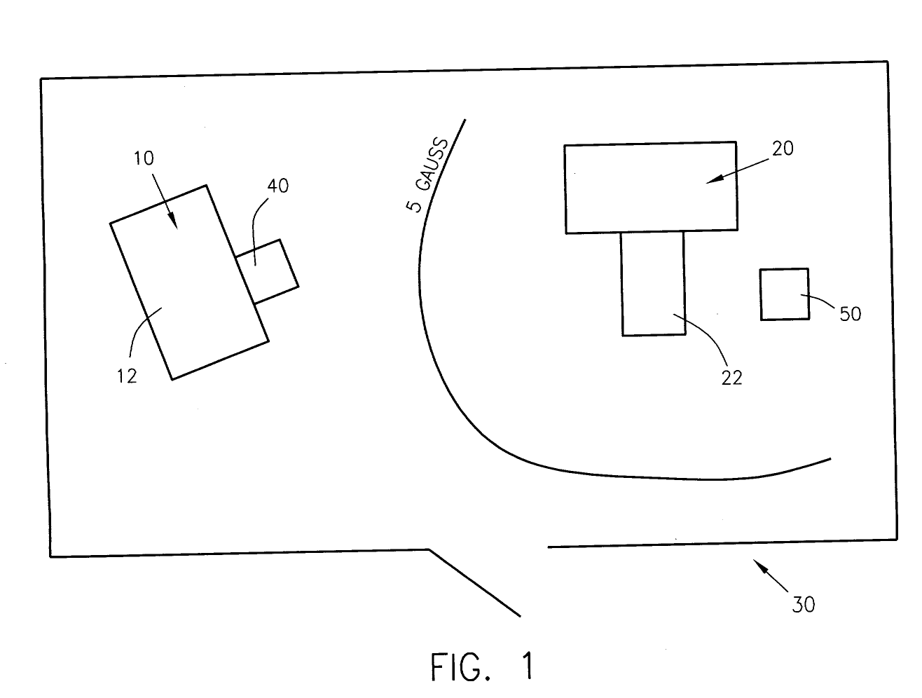

As shown in FIG. 1, a preferred embodiment of the present invention includes an angiographic/fluoroscopic (“angio”) imaging system 10 and a magnetic resonance (“MR”) imaging system 20 located in a common room or suite 30 (sometimes referred to as an XMR suite). Although the present invention is discussed herein in terms of a single or common suite, the invention contemplates that the two imaging systems may also be located in adjoining or relatively adjacent suites.

The imaging systems 10, 20 each include a bed 12, 22, respectively on which a patient being imaged lies. An angiographic injector 40, such as the Mark V ProVis® injector sold by Medrad, Inc., the assignee of the present application, is positioned adjacent to the angio system 10 on a separate stand or is preferably attached to the bed 12, for easy access to the patient for a contrast injection. A MR injector 50, such as the Spectris® injector sold by Medrad, Inc., may also be positioned adjacent to the MR system 20 for contrast injections.

Because angio injectors are not currently designed to also be compatible with MR systems (due, for example, to the permanent-magnet DC motors, AC power supply and RF emissions attendant with or required for those injectors), the angio injector 40 must be positioned sufficiently remote (usually within 5-20 feet) from the MR system to preclude substantial interference between and among the MR system 20, the angio injector 40 and the angio system 10. Typically, as shown in FIG. 1, the angio injector 40 should be positioned outside of the 5 Gauss field line developed by the MR system.

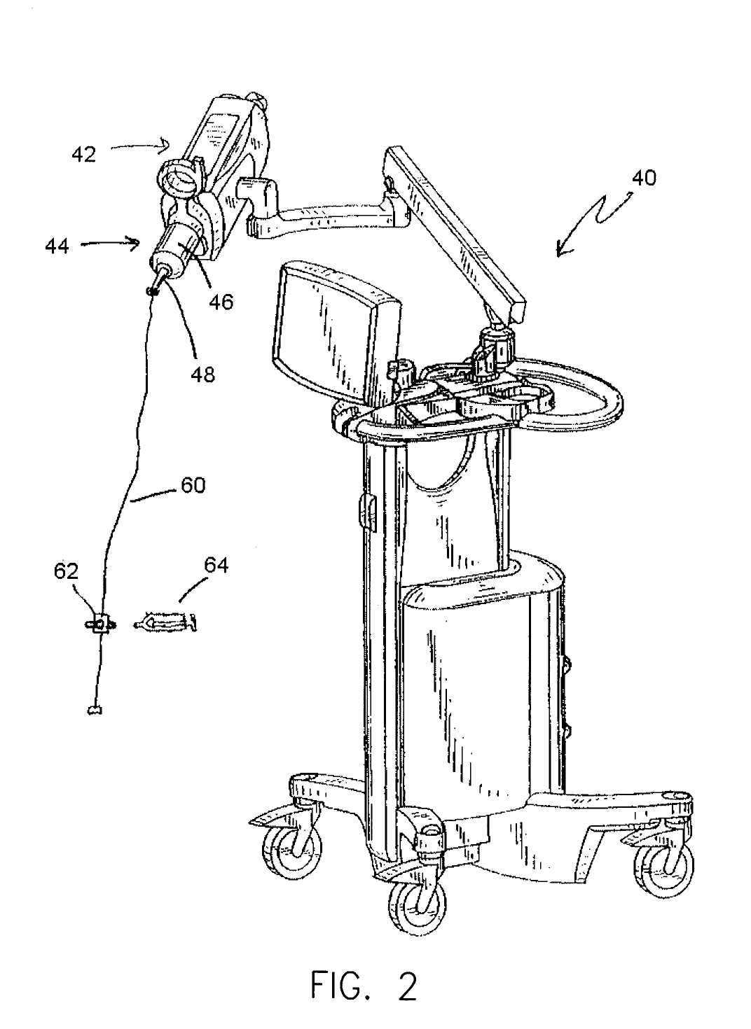

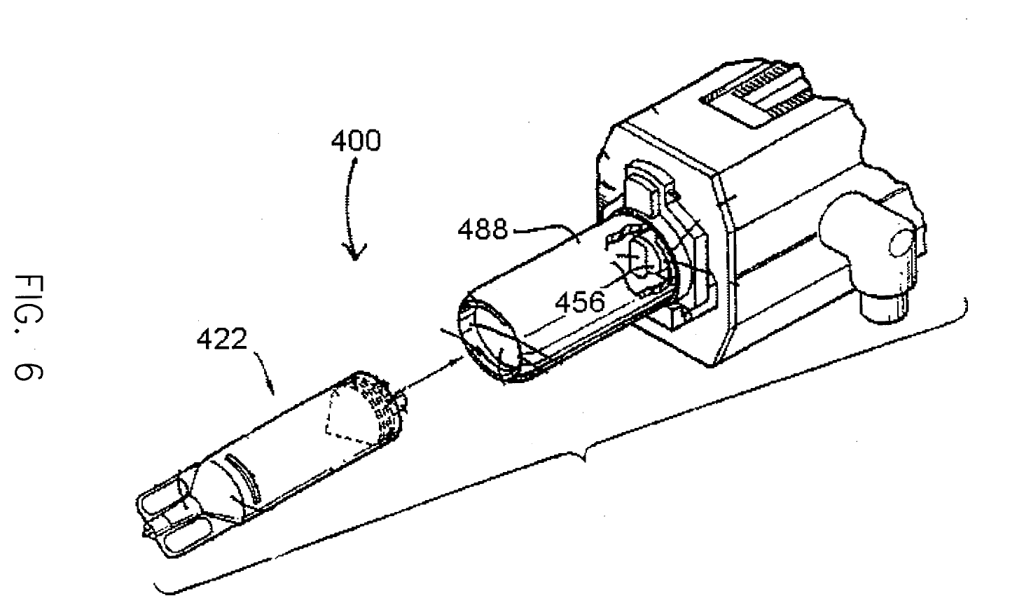

A stand-alone angiographic injector is shown in FIG. 2. This injector may be used with or in the present invention as the angio injector 40 shown in FIG. 1. The injector 40 includes an injector head 42 having a pressure jacket assembly 44 mounted thereon. The injector head preferably includes a drive member or piston (see piston 456 in FIG. 6) for engaging a plunger in a syringe. The pressure jacket assembly 44 includes a pressure jacket 46 and a removably syringe 48 disposed therein. As described in more detail below, the syringe 48 preferably is rear- or breech-loaded into the rear end of the pressure jacket 46, and then the combined pressure jacket assembly 44 is front-loaded and connected to the injector head 42.

After the pressure jacket assembly 44 is front-loaded onto the injector head 42, a patient connector tube 60, which preferably includes a three-way or four-way stop-cock 62, is connected between the fluid discharge outlet of the syringe 48 and a catheter (not shown) placed in the patient (not shown). In many applications, a hand-held syringe 64 may be connected to a port of the stopcock 62 to, for example, injector a therapeutic drug or conduct a test injection of a small amount of contrast.

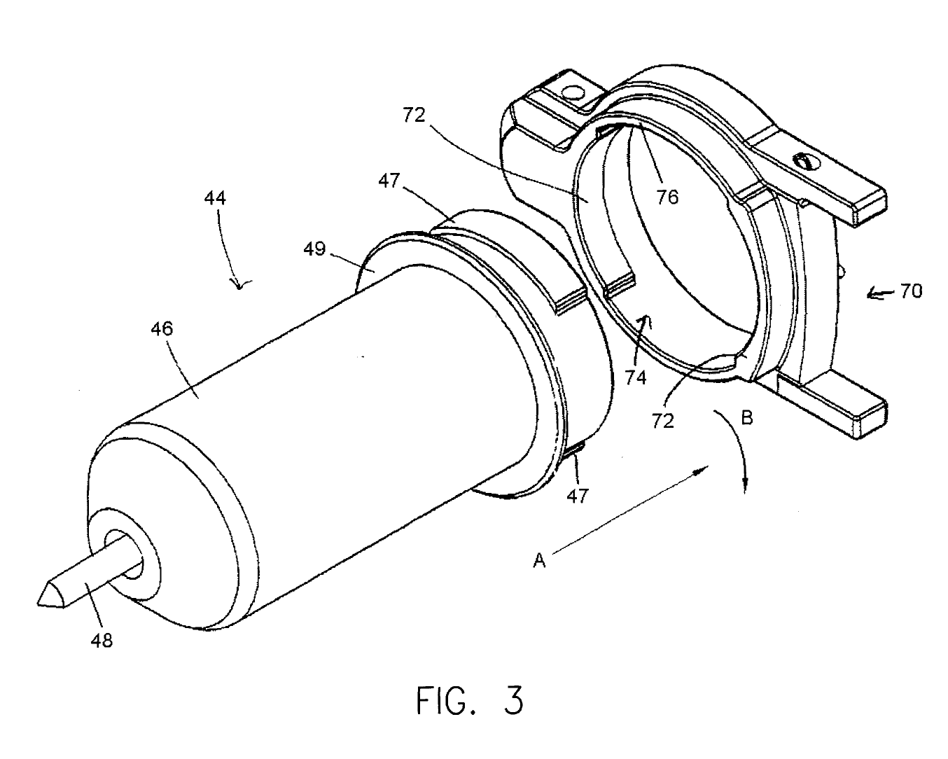

A preferred embodiment of the pressure jacket assembly 44 and an injector head retaining mechanism 70 are shown in FIG. 3. The retaining mechanism 70 may be removably or permanently mounted on the injector head 42 (see FIG. 2) for retaining the pressure jacket assembly 44 thereon. The pressure jacket assembly 44 is preferably releasably or removably connected to the retaining mechanism 70 by means of a bayonet connection. To implement the bayonet connection, the retaining mechanism 70 preferably includes two retaining flanges 72 formed in an interface 74 therein. The pressure jacket 46 preferably includes two complementary mounting flanges 47 formed thereon for mating with the retaining flanges 72. Further, the pressure jacket 46 may include a sealing or biasing flange 49 for abutting a peripheral edge 76 of the retaining mechanism 70 when the pressure jacket 46 or pressure jacket assembly 44 is mounted thereon.

To prepare the injector 40 for an injection procedures, the pre-loaded or prefilled syringe 48 is rear- or breech-loaded into the pressure jacket 46 and the combined pressure jacket assembly 44 is inserted into the retaining mechanism 70 (see Arrow A in FIG. 3) and then rotated (see Arrow B) to engage the two mounting flanges 47 with the two retaining flanges 72. A similar bayonet apparatus for front-loading a syringe onto an injector is disclosed in U.S. Pat. No. 5,383,858, the disclosure of which is incorporated herein by reference. In addition, a syringe that may be used with the pressure jacket 46 is disclosed in U.S. Pat. No. 4,677,980, the disclosure of which is incorporated herein by reference.

The pressure jacket 46, including the flanges 47 and the biasing flange 49 may be formed of a polymeric material, including polycarbonate. In a preferred embodiment, however, the flanges 47 and/or the biasing flange 49 may be part of a separate sleeve or adapter that is attached or connected to the rear end of the pressure jacket 46. In a most preferred embodiment, the sleeve is formed of a non-ferromagnetic material, such as stainless steel, so as not to be drawn into the MR system 20 by the strong magnetic field generated thereby. Preferably, the sleeve is press-fit onto the rear end of the pressure jacket 46 and most hold approximately 2400 lbs.-force to accommodate the rated fluid pressure generated by the angio injector 40. In addition, an adhesive, such as Scotch-Weld™ epoxy adhesive provided by 3M, may be placed between the pressure jacket 46 and the sleeve.

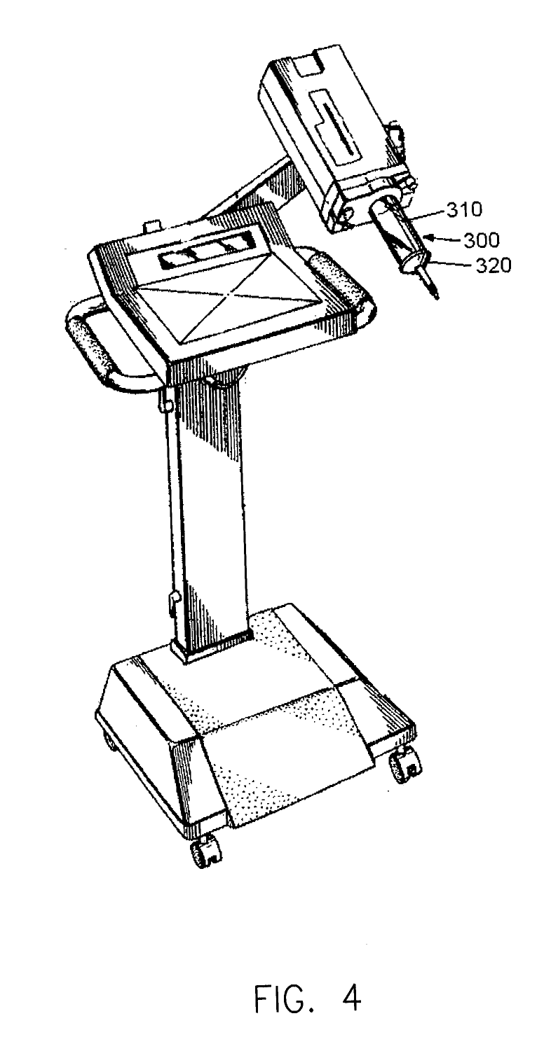

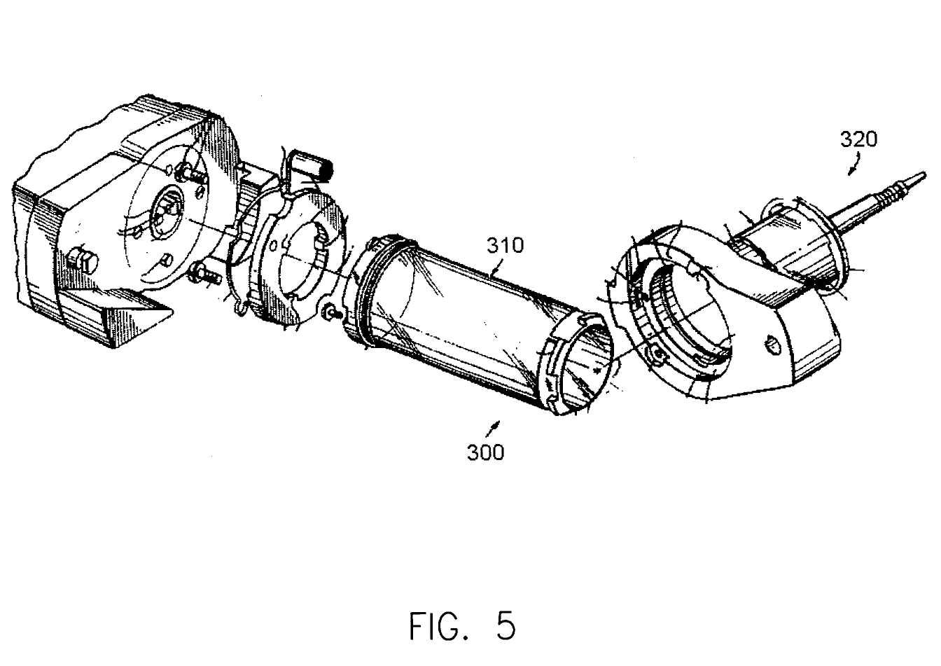

In addition to the pressure jacket assembly 46 discussed above, other pressure jacket assemblies and syringes may be used with or in the present invention. For example, as shown in FIGS. 4 and 5, the front-loading pressure jacket assembly 300, including the pressure jacket 310 and the syringe 320, may be used. In this embodiment, the syringe 320 includes structure on a front end thereof that mates with corresponding structure on the front end of the pressure jacket 310 to lock the syringe thereto. The structure and operation of the pressure jacket assembly 300 is fully described in U.S. Pat. No. 5,300,031, the contents of which are incorporated herein by reference.

Further, the pressure jacket assembly 400 shown in FIG. 6 may be used with or in the present invention. In this embodiment, the syringe 422 includes structure on the front end thereof that mates with corresponding structure on the interior of the front end of the pressure jacket 488. The structure and operation of the pressure jacket assembly 400 is fully described in U.S. Pat. No. 5,383,858, the contents of which are incorporated herein by reference.

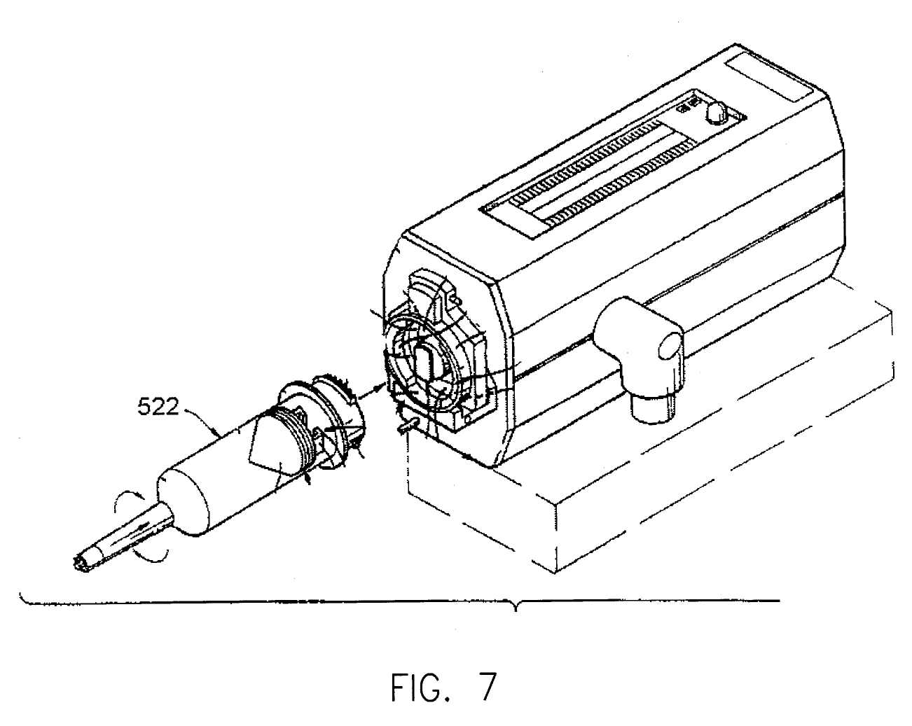

In another embodiment shown in FIG. 7, a front-loading syringe 522 (without an enclosing pressure jacket) may be used with or in the present invention. The structure and operation of the front-loading syringe 522 is fully described in U.S. Pat. No. 5,383,858, the contents of which are incorporated herein by reference.

In a preferred embodiment of the present invention, a patient (not shown) is brought into the XMR suite shown in FIG. 1 and placed on the table 12 of the angio system 10. A contrast fluid container, such as a syringe, is placed in a pressure jacket that is mountable (see assembly 44) or is already mounted (see assemblies 300, 400) on the angio injector 40 or is mounted directly (see syringe 522) on the angio injector 40. A connecting tube 60 is connected to the syringe 48, 320, 422, 522, and contrast is forced through the tubing to remove the air therefrom. After the air is purged from the connecting tube 60, the end of the tube 60 is connected to the catheter (not shown) in the patient. The stopcock 62, if provided with the connecting tube 60, is activated to open the fluid path from the syringe 48, 320, 422, 522 to the patient. A contrast injection is performed and one or more images of the patient are generated by the angio system 10.

After the angio procedure is completed, the stopcock (if provided) is activated to close the fluid path to the patient. The pressure jacket assembly 44 or the syringe 320, 422, 522 may be removed from the injector (with the connecting tube 60 preferably still connected between the syringe 48, 320, 422, 522 and the patient) and placed with or adjacent to the patient (e.g., on a bed, table or gurney). The patient (and the pressure jacket assembly 44 or the syringe 320, 422, 522) is then moved to the adjacent MR system 20 for an MR imaging procedure. After the MR imaging procedure is completed, the patient (including the pressure jacket assembly 44 or the syringe 320, 422, 522) may then be returned to the adjacent angio system 10 for a new angio procedure.

To conduct the new angio procedure, the pressure jacket assembly 44 or the syringe 320, 422, 522 is re-connected to the injector 40, the stopcock (if provided) is activated to open the fluid path to the patient, the contrast injection is performed, and one or more angio images are generated by the angio system 10. The first and new angio images and the MRI images may then be analyzed to diagnose the patient's condition or illness.

In other embodiments, additional imaging systems, such as ultrasound, CT and PET imaging systems, may be placed in the XMR suite or adjacent suites for diagnostic and/or therapeutic purposes. Further, if a contrast fluid and is compatible with more than one imaging procedure, the present invention may be used to provide a removable contrast fluid container that may be compatible with and used on injectors designed for or dedicated to the respective imaging systems. Moreover, if the removable contrast fluid container is not compatible with more then one injector system, a second or additional removable contrast fluid or drug containers that do fit on or are otherwise compatible with the other injector systems may be designed in accord with the present invention, connected to the patient (via a common connector tube or manifold system, as is known in the art) and movable therewith between and among the various imaging systems.

The foregoing description and accompanying drawings set forth the preferred embodiments of the invention at the present time. Various modifications, additions and alternative designs will, of course, become apparent to those skilled in the art in light of the foregoing teachings without departing from the scope of the invention. The scope of the invention is indicated by the following claims rather than by the foregoing description. All changes and variations that fall within the meaning and range of equivalency of the claims are to be embraced within their scope.

What is claimed is:

1. A method of performing multiple imaging procedures on a patient using at least a first imaging system, a second imaging system and an injector system comprising a removable contrast fluid container, the method comprising:
injecting contrast fluid from the removable contrast fluid container into the patient;
generating at least a first contrast-enhanced image of the patient using the first imaging system;
disconnecting the removable contrast fluid container from the injector system;
moving the patient and the removable contrast fluid container from the first imaging system to the second imaging system;
generating an image of the patient using the second imaging system;
moving the patient and the removable contrast fluid container from the second imaging system to the first imaging system;
connecting the removable contrast fluid container to the injector system;
injecting contrast fluid from the removable contrast fluid container into the patient; and
generating at least a second contrast-enhanced image of the patient using the first imaging system.

2. The method of claim 1 wherein the first imaging system comprises an angiographic imaging system.

3. The method of claim 1 wherein the second imaging system comprises a magnetic resonance imaging system.

4. The method of claim 1 wherein the removable contrast fluid container comprises a syringe.

5. The method of claim 4 wherein the removable contrast fluid container further comprises a pressure jacket at least partially surrounding the syringe.

6. The method of claim 1 wherein the removable contrast fluid container comprises a bulk contrast container.

7. The method of claim 1 wherein the injector system comprises an angiographic injector system.

8. The method of claim 7 wherein the angiographic injector system is capable of injecting the contrast fluid at a pressure of approximately 1200 p.s.i.

9. The method of claim 1 wherein the removable contrast fluid container does not substantially interfere with an electromagnetic field of the magnetic resonance imaging system.

10. The method of claim 1 wherein the removable contrast fluid container is formed of non-ferromagnetic material.

11. The method of claim 1 wherein a fluid path is connected to the removable contrast fluid container and a catheter placed in the patient.

12. The method of claim II, further comprising a stopcock disposed in the fluid path.

13. The method of claim 12, further comprising the step of activating the stopcock to close the fluid path to the patient.

14. The method of claim 13 wherein the stopcock is activated prior to disconnecting the removable contrast fluid container from the injector system.

15. The method of claim 12, further comprising the step of activating the stopcock to open the fluid path to the patient.

16. The method of claim 15 wherein the stopcock is activated after connecting the removable contrast fluid container to the injector system.

17. The method of claim 1 wherein the first imaging system comprises a computed tomography imaging system.

18. The method of claim 1 wherein the first imaging system and the second imaging system are located in a single suite.

19. The method of claim 1 wherein the injector system is connectively associated with the first imaging system.

20. A method of performing multiple imaging procedures on a patient using at least a first imaging system, a second imaging system and an injector system comprising a removable contrast fluid container, the method comprising:
injecting contrast fluid from the removable contrast fluid container into the patient;
generating at least a first contrast-enhanced image of the patient using the first imaging system;
disconnecting the removable contrast fluid container from the injector system;
moving the patient and the removable contrast fluid container from the first imaging system to the second imaging system;
generating an image of the patient using the second imaging system; and
moving the patient and the removable contrast fluid container from the second imaging system to the first imaging system.

21. The method of claim 20, further comprising:
connecting the removable contrast fluid container to the injector system;
injecting contrast fluid from the removable contrast fluid container into the patient; and
generating at least a second contrast-enhanced image of the patient using the first imaging system.

22. The method of claim 20, further comprising:
providing a third imaging system; and
generating an image of the patient using the third imaging system.

23. The method of claim 20, further comprising:
providing a fluid path comprising a stopcock;
connecting one end of the fluid path to the removable contrast fluid container and the other end of the fluid path to a catheter disposed in the patient;

24. The method of claim 23, further comprising the step of activating the stopcock to close the fluid path to the patient.

25. The method of claim 24 wherein the stopcock is activated prior to disconnecting the removable contrast fluid container from the injector system.

26. The method of claim 20 wherein the removable contrast fluid container comprises a syringe.

27. The method of claim 26 wherein the removable contrast fluid container further comprises a pressure jacket at least partially surrounding the syringe.

28. An injector system comprising:
an injector comprising:
a housing;
a drive member at least partially disposed within the housing and operable to engage a plunger of a syringe; and
a retaining mechanism associated with the housing, the retaining mechanism comprising at least one retaining flange; and
a front-loading pressure jacket assembly comprising:
a pressure jacket having a front end and a rear end, the rear end adapted to receive a syringe inserted therein; and
a mounting member operably associated with the rear end of the pressure jacket, the mounting member comprising at least one mounting flange adapted to releasably engage the at least one retaining flange of the retaining mechanism, the mounting member formed of a non-ferromagnetic material; and
a syringe comprising:
a body defining a rear end and a fluid discharge end; and
a plunger movably disposed within the body.

29. The injector system of claim 28 wherein the at least one retaining flange comprises two retaining flanges and the at least one mounting flange comprises two mounting flanges.

30. The injector system of claim 28 wherein the mounting member is formed of stainless steel.

31. The injector system of claim 28 wherein the mounting member is press fit onto the pressure jacket.

32. The injector system of claim 28, further comprising an adhesive for adhering the mounting member to the pressure jacket.

33. The injector system of claim 28, further comprising a fluid path having a first end connectable to the fluid discharge end of the syringe and a second end connectable to a catheter in a patient.

34. The injector system of claim 33 wherein the fluid path comprises a stopcock.

35. The method of claim II wherein the fluid path remains connected to the removable contrast fluid container and the catheter when the removable contrast fluid container is disconnected from the injector system.

36. The injector system of claim 28 wherein the pressure jacket is formed of polymeric material.

37. The injector system of claim 36 wherein the polymeric material comprises polycarbonate.

38. A system for performing multiple imaging procedures on a patient, the system comprising:
a first imaging system;
a second imaging system; and
an injector system comprising:
an injector comprising:
a housing; and
a retaining mechanism associated with the housing, the retaining mechanism comprising at least one retaining flange; and
a front-loading pressure jacket assembly comprising:
a pressure jacket having a front end and a rear end, the rear end adapted to receive a syringe inserted therein; and
a mounting member operably associated with the rear end of the pressure jacket, the mounting member comprising at least one mounting flange adapted to releasably engage the at least one retaining flange of the retaining mechanism, the mounting member formed of a non-ferromagnetic material; and
a syringe comprising:
a body defining a rear end and a fluid discharge end; and
a plunger movably disposed within the body.

39. The system of claim 38 wherein the first imaging system comprises an angiographic imaging system, the second imaging system comprises a magnetic resonance imaging system and the injector system comprises an angiographic injector system.

40. The system of claim 39, further comprising a fluid path having a first end connectable to the fluid discharge end of the syringe and a second end connectable to a catheter in a patient.
